(19) United States
(12) Patent Application Publication
(10) Pub. No.: US 2008/0102107 A1
(43) Pub. Date: May 1, 2008

(54) Compositions and methods for transdermal joint pain therapy

(75) Inventors: Gary Lewellyn, Oklahoma City, OK (US); J. Calvin Johnson, Edmond, OK (US); John Lassiter, Del City, OK (US)

Correspondence Address: PATTON BOGGS, LLP, 2001 ROSS AVENUE, SUITE 3000, DALLAS, TX, 75201, US

(73) Assignee: Valex Pharmaceuticals, LLC, Oklahoma City, OK (US)

(21) Appl. No.: 11/305,552

(22) Filed: Dec. 16, 2005

(51) Int. Cl. A61K 31/728; A61K 31/7008; A61K 31/198

(52) U.S. Cl. 424/448; 514/54; 514/62; 514/563

(57) ABSTRACT

Compositions suitable for use in treating pain and increasing range of motion of an affected joint of an animal, particularly a human. The compositions contain a transdermal delivery formulation for delivering effective amounts of glutamine, hyaluronic acid, methysulfonylmethane, and glucosamine to the affected joint. The compositions are applied topically to the skin area adjacent to the affected joint. Methods for treating pain and increasing range of motion that use the transdermal joint pain therapy compositions are also provided.

### FIELD OF THE INVENTION

This invention relates to compositions and methods suitable for use in transdermal joint pain therapy, and in particular topical compositions that effectively reduce pain and swelling and increase the range of motion of an affected joint.

### Problem

It is a problem in the field of joint therapy to reduce the pain and swelling of affected joints of animals, in particular humans. Pain is defined as an unpleasant sensory and emotional experience, which is primarily associated with tissue damage or described in terms of such damage, or both. Pain reflects both a sensory experience and the individual's affective and cognitive responses. Research has shown that pain impacts not only physical function, but also psychological, social, and role functioning of humans as well. Research also demonstrates that sleep disruption of even a single night's duration due to persistent pain affects not only cognitive function, but also has a significant impact on metabolic and physiological function as well, particularly in elderly patients. Chronic joint pain and restricted joint motion are the leading causes of limitation of activity and disability among adults in the U.S. In 2001, one-third of all American adults reported chronic joint symptoms or physician diagnosed arthritis. By the year 2030, researchers estimate that 41 million Americans over the age of 65 will suffer chronic joint symptoms. It is also clear that a growing number of individuals under the age of 65 are also reporting disability and chronic pain associated with arthritis.

The most common approach to treatment of chronic joint pain is pharmacologic in the form of both prescribed and over-the-counter non-steroidal anti-inflammatory agents (NSAIDs) and prescription Cox 2 Inhibitors. In more recent years, intra-articular injection of hyaluronic acid has been used for treating joint pain in subjects. While NSAIDs have been shown to effectively control certain symptoms in many instances, their use is also associated with substantial risk of gastrointestinal disturbance and potential cardiovascular damage with chronic use. Cox 2 Inhibitor usage has been significantly reduced by recent scientific revelations of potential cardiovascular damage attributed to the brand names Vioxx® and Bextra®. Injectible hyalgan (hyaluronic acid) has been restricted by cost, insurance reimbursement, and local injections site irritation. As an overall consequence of these issues, the ability to effectively treat the patient has been significantly impacted.

More recently, the concept of oral administration of hyaluronic acid has gained in popularity for joint pain. One problem associated with this concept is that the amount of hyaluronic acid ingested does not equal the amount of hyaluronic acid delivered to the affected joint area because of its widespread dissemination throughout the body and its bioavailability after ingestion by a subject.

Also recently, treatment procedures for painful joints have included administration of topical formulations that include glucosamine, glutamine, and dimethyl sulfone, but that do not contain an effective transdermal component to deliver the composition to the affected joint. Other topical formulations include hyaluronic acid for use as a penetrating agent that is combined with other compounds for treating cancer through the lymph system. Other topical compositions have been formulated using hyaluronic acid as the transport mechanism for treatment of skin conditions. Yet still other topical compositions utilize hyaluronic acid as a drug delivery system for delivering interacting components related to fusion proteins.

Information relevant to attempts to address these problems can be found in U.S. Pat. Nos. 5,792,753 issued Aug. 11, 1998 to Falk, et al.; U.S. Pat. No. 6,218,373 issued Apr. 17, 2001 to Falk, et al.; U.S. Pat. No. 6,645,510 issued Nov. 11, 2003 to Coury, et al.; U.S. Pat. No. 6,841,173 issued Jan. 11, 2005 to Reynolds; U.S. Pat. No. 4,808,576 issued Feb. 28, 1989 to Schultz, et al.; U.S. Pat. No. 6,733,753 issued May 11, 2004 to Boone, et al.; U.S. Pat. No. 6,579,543 issued Jun. 17, 2003 to McClung; and U.S. Pat. App Nos. US2005/0025840 to Reynolds filed Aug. 31, 2004; US2004/0019011 to Falk et al. filed Jul. 28, 2003; US2004/0122105 to Bettle, III, et al. filed Sep. 22, 2003. Nonetheless, each one of these references suffers from one or more of the following disadvantages: ineffective transdermal delivery systems, treatment for skin diseases and not joint pain, and limited or uncertain bioavailability of the active components to the target area.

Therefore, there is a need for a therapeutical composition that is applied topically, not orally or through injection, to the skin area adjacent to an affected joint of a subject that reduces the overall pain and increases the activities and range of motion of the affected joint of a subject.

### Solution

The above described problems are solved and a technical advance is achieved by the present compositions and methods of transdermal joint pain therapy. The present compositions and methods of transdermal joint pain therapy eliminates both the gastrointestinal and cardiovascular concerns associated with chronic use of oral NSAIDs and Cox 2 Inhibitors, as well as site irritation from injections. The present transdermal joint pain therapy compositions are absorbed significantly faster, have no known side effects, and can be used to treat larger affected areas with rapid, effective delivery of therapeutic formulations to an a targeted joint area to reduce pain and enhance range of motion.

The advantage of the present compositions and methods of transdermal joint pain therapy is that the effective components are absorbed through the skin area adjacent to a targeted joint for delivery of the effective components to the joint without side effects and without having to treat larger affected areas. The present compositions and methods of transdermal joint pain therapy eliminates the concerns associated with the use of traditional NSAIDs, COX 2 Inhibitors, and injectible hyalgan. The present composition for transdermal joint pain therapy includes glutamine, hyaluronic acid, methylsulfonylmethane, glucosamine, and a transdermal delivery agent that are mixed together to form a gel or cream that is applied topically to the skin adjacent to a targeted joint area.

### SUMMARY

A transdermal joint pain therapy composition including from about 2.5% to about 15%, based on the total weight of the transdermal joint pain therapy composition, of glutamine; from about 0.04% to about 0.5%, based on the total weight of the transdermal joint pain therapy composition, of hyaluronic acid; from about 2.5% to about 10.0%, based on the total weight of the transdermal joint pain therapy composition, of methylsulfonylmethane; and from about 70% to about 95%, based on the total weight of the transdermal joint pain therapy composition, of a transdermal delivery agent. Preferably, the transdermal delivery agent includes: at least one or more compounds selected from the group consisting of cetyl alcohol, stearic acid, glyceryl monostearate, isopropyl myristate, lecithin, butylated hydroxyl toluene, simethicone, urea, potassium sorbate, sodium hydroxide, polyoxyl 40 stearate, EDTA disodium, and water.

Preferably, the transdermal joint pain therapy composition further includes from about 0.1% to about 15.0%, based on the total weight of the transdermal joint pain therapy composition, of glucosamine HCl. Preferably, the transdermal joint pain therapy composition further includes from about 5.0% to about 20.0%, based on the total weight of the transdermal joint pain therapy composition, of lecithin. Preferably, the transdermal joint pain therapy composition further includes from about 5.0% to about 20.0%, based on the total weight of the transdermal joint pain therapy composition, of propylene glycol. Preferably, the transdermal delivery agent is a gel, a cream, and an ointment. Preferably, the transdermal delivery agent further includes a paraben based preservative. Preferably, the paraben-based preservative includes from about 0.6% to about 0.9%, based on the total weight of the transdermal joint pain therapy composition, of a mixture selected from the group consisting of phenoxyethanol, methylparaben, propylparaben, butylparaben, and isobutylparaben.

### DETAILED DESCRIPTION OF THE PREFERRED EMBODIMENTS

All weights, measurements and concentrations herein are measured at 25 degrees centigrade on the composition in its entirety, unless otherwise specified. Unless otherwise indicated, all percentages of compositions referred to herein are weight percentages of the total composition (i.e. the sum of all components present) and all ratios are weight ratios. Unless otherwise indicated, all polymer molecular weights are weight average molecular weights. Unless otherwise indicated, the content of all literature sources referred to within this text are incorporated herein in full by reference. Except where specific examples of actual measured values are presented, numerical values referred to herein should be considered to be qualified by the word “about”.

The present compositions and methods for transdermal joint pain therapy are for treating pain and increasing the range of motion of affected joints of an animal, particularly a human. Without limitation, these joint pains include: shoulder pain, such as AC arthrosis (bursitis, rotator cuff tendonitis); elbow pain, such as medial and lateral Epicondylitis (tennis elbow, golfer's elbow); wrist pain, such as extension and flexor tendonitis; De Quervain's Tenosynovitis (tendonitis); finger problems; hip pain, caused by a “snapping hip;” Trochanteric Bursitis; knee pain; Patellar Athrosis; Post-traumatic patellofemoral pain; Patellar Tendonitis (runner's knee, jumper's knee); Plica; Apophysitis (Osgood-Schlatter) (growing pains); leg pain, such as Gastrocnemius Strain (calf strain); Achilles Tendonitis; ankle pain, such as acute or chronic ankle sprain; foot pain, such as retrocalcaneal Bursitis; Sever's Disease; Plantar Fasciitis; Posterior Tibial Tendonitis (shin splint); Metatarsalgia (toe pain); Turf Toe; Sesamoid Dysfunction; back pain, such as Lumbar Strain; SI pain (sciatica pain); cervical pain and strain; and Trapezius Trigger Points (pinched nerve).

The present transdermal joint pain therapy composition preferably includes a transdermal base component and a joint pain therapy component.

### Transdermal Base Component

In one embodiment, the transdermal base according to the present transdermal joint pain therapy composition may include one or more of the following: a solvent, a preservative, a humectant, a stabilizing agent, a thickening agent, an emulsifying agent, an anti-oxidant, mold and yeast growth inhibitors, and an alkalizing agent.

In one embodiment, the present transdermal joint pain therapy composition includes a solvent, preservative, or humectant, such as propylene glycol that has the chemical formula C₃H₈O₂, (CAS # 57-55-6). Propylene glycol must be heated or briskly shaken to produce a vapor. Propylene glycol is a humectant and is used to transport active ingredients into the skin of a subject. In one embodiment, the present transdermal joint pain therapy composition contains propylene glycol in an amount preferably between 0% to 25% by weight, based on the total weight of the transdermal joint pain therapy composition. A non-limiting exemplary chemical structure for propylene glycol is:

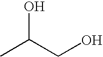

Other known preservative agents that can be used and include, but are not limited to, hydroquinone, pyrocatechol, resorcinol, 4-n-hexyl resorcinol, captan (i.e., 3a,4,7,7a-tetrahydro-2-((trichloromethyl)thio)-1H-isoindole-1,3(2H)-dione), benzethonium chloride, benzoic acid, benzyl alcohol, cetylpyridinium chloride, chlorobutanol, dehydroacetic acid, o-phenylphenol, phenol, phenylethyl alcohol, potassium benzoate, potassium sorbate, sodium benzoate, sodium propionate, sorbic acid, thimerosal, thymol, chlorothymol, alcohols, chlorobutanol, phenoxy-2-ethanol, benzyl alcohol, .beta.-phenylethyl alcohol, chlorhexidine, 6-acetoxy-2,4-dimethyl-m-dioxane 2,4,4′trichloro-2′-hydroxy-diphenylether, imidizoldinylether urea compound, bromo-2-nitropropanediol-1,3,5-bromo-5-nitrol-1,3 dioxane 2-methyl 1-4-isothiazolin-3-one and 5 chloro derivative 1-(3-chloroallyl)-3,5,7-triazo 1-azoniaadamantane chloride (Dowicil 200), phenylmercuric compounds such as phenylmercuric borate, phenylmercuric nitrate and phenylmercuric acetate, formaldehyde, formaldehyde generators such as the preservatives Germall II™ and Germall 115™ (imidazolidinyl urea, available from Sutton Laboratories, Charthan, N.J.), Germaben, Germaben I, Germaben II, morpholines, salicylic and benzoic acids, sodium and potassium iodides, flucytosine, 5-flucytosine, griseofulvin, terbinafine, cidofovir, famicoclovir, valacyclovir, echinocandins, pneumocandins, pradimicins, benanomicins, nikkomycins, amorolfine, polyoxins, duanorubicin citrate, doxorubicin hydrochlolide, tolnaftate, ciclopirox, butenafine, and ergestrol biosynthesis inhibitors.

Other preservative and emollients include hydroxypivalyl hydroxypivalate and its alkoxylated derivatives, TMPD, TMPD alkoxylates, ethanol, isopropanol, butanol, 1,2-cyclohexanedimethanol, 1,4-cyclohexanedimethanol, HPHP glycol, 1,2-hexanediol, ethylene glycol butyl ether, hexylene glycol, isoprene glycol, sorbitan ethoxylates, 2-butoxyethanol, C₆-C₁₂ diols/triols and ester diols/triols and their alkoxylated derivatives, glycol ethers, and mixtures thereof.

In one embodiment, the present transdermal joint pain therapy composition includes an active surface agent to stabilize the emulsion and to increase its ability to retain large quantities of water, such as cetyl alcohol that has the chemical formula of C₁₆H₃₄O, (CAS # 36653-82-4); stearyl alcohol that has the chemical formula of CH₃(CH₂)₁₆CH₂OH, (CAS # 112-92-5); and stearic acid that has the chemical formula of CH₃(CH₂)₁₆COOH, (CAS # 57-11-4). Cetyl alcohol is a high molecular straight chain primary alcohol that functions as an emulsifying and thickening agent for the present transdermal joint pain therapy composition. Stearyl alcohol is a synthetic fatty alcohol that is equivalent chemically and physically to natural alcohols obtained from oleochemical sources, such as coconut and palm kernel oil. Stearyl alcohol is also used as an emulsifying agent for the present transdermal joint pain therapy composition. These emulsifying agents help to mix two liquids that are otherwise immiscible. In addition, they also are used as thickeners for the present transdermal joint pain therapy composition. Due to the surfactant properties of these compounds, they also reduce the surfactant tension of a liquid, thus allowing for easier spreading of the present transdermal joint pain therapy composition on the skin of a subject. In one embodiment, the present transdermal joint pain therapy composition contains cetyl alcohol in an amount preferably between 0% to 5% by weight, based on the total weight of the transdermal joint pain therapy composition; stearyl alcohol in an amount preferably between 0% to 2% by weight, based on the total weight of the transdermal joint pain therapy composition; and stearic acid in an amount preferably between 0% to 7.5% by weight, based on the total weight of the transdermal joint pain therapy composition. A non-limiting exemplary chemical structure for cetyl alcohol is:

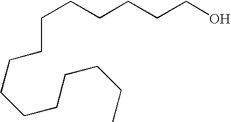

A non-limiting exemplary chemical structure for stearyl alcohol is:

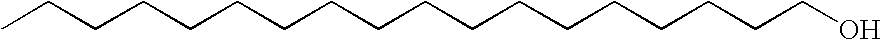

A non-limiting exemplary chemical structure for stearic acid is:

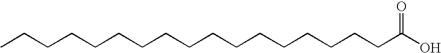

In one embodiment, the present transdermal joint pain therapy composition includes a thickening agent and an emulsifying agent, such as glyceryl monostearate that has the chemical formula of CH₃(CH₂)₁₆COOCH₂CHOHCHCH₂OH, (CAS # 31566-31-1) and polyoxyl 40 stearate that has the chemical formulas of HO(CH₂CH₂O)ₙH (free polyol); RCOO(CH₂CH₂O)ₙH (monoester); and RCOO(CH₂CH₂O)ₙOCR (diester). Polyoxy 40 stearate, also known as polyoxyethylene (40) stearate, is composed of mixed polyoxy diols (an average of 40 polymers) and mono and di-esters of commercial stearic acid. In one embodiment, the present transdermal joint pain therapy composition contains glyceryl monostearate in an amount preferably between 0% to 7.5% by weight, based on the total weight of the transdermal joint pain therapy composition and polyoxyl 40 stearate in an amount preferably between 0% to 7.5% by weight, based on the total weight of the transdermal joint pain therapy composition. A non-limiting exemplary chemical structure for glyceryl monostearate is:

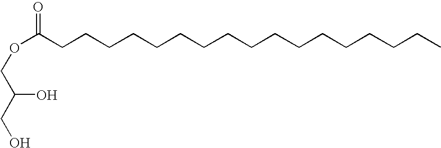

The present transdermal joint pain therapy composition may further include additional thickening agents. Generally, these thickening agents also provide an emulsion stabilizing function to the present transdermal joint pain therapy composition. An exemplary thickening agent is carbomer and water. The present transdermal joint pain therapy composition may contain carbomers available from B.F. Goodrich under the tradename, “Carbopol ETD 2020” and water in an amount preferably between 10% to 20% by weight. In one embodiment, the present transdermal joint pain therapy composition contains an additional thickener, tridecyl stearate, in an amount preferably between 2.5% to 7.5%.

Additional exemplary thickening agents nonexclusively include acrylate copolymers, hydroxyethylcellulose modified with cetyl ether groups available from Hercules under the tradename, “Natrosol Plus”, polyvinylmethyl ether/maleic anhydride (PVM/MA) decadiene crosspolymer available from International Specialty Products under the tradename, “Stabileze QM,” and copolymers and mixtures thereof, with carbomers being preferred. Examples of suitable acrylate copolymers nonexclusively include acrylate copolymers available from Rohm & Haas under the tradename, “Aculyn 33,” acrylates/aminoacrylates copolymer available from National Starch & Chemical Company under the tradename, “Structure Plus,” acrylates/steareth-20 itaconate copolymer available from National Starch & Chemical Company under the tradename, “Structure 2001,” acrylates/ceteth-20 itaconate copolymer available from National Starch & Chemical Company under the tradename, “Structure 3001,” acrylates/steareth-20 methacrylate copolymer available from Rohm & Haas under the tradename, “Aculyn 22,” and copolymers and mixtures thereof.

The present transdermal joint pain therapy composition may also include additional thickening agents such as alkyl silicones, alkyl trimethylsilanes, beeswax, behenyl behenate, behenyl benzoate, C₂₄-C₂₈alkyl dimethicone, C₃₀alkyl dimethicone, cetyl methicone, stearyl methicone, cetyl dimethicone, stearyl dimethicone, cerotyl dimethicone, candelilla wax, carnauba, synthetic carnauba, PEG-12 carnauba, cerasin, hydrogenated microcrystalline wax, jojoba wax, microcrystalline wax, lanolin wax, ozokerite, paraffin, synthetic paraffin, cetyl esters, behenyl behenate, C₂₀-C₄₀ alkyl behenate, C₁₂-C₁₅ lactate, cetyl palmitate, stearyl palmitate, isosteryl behenate, lauryl behenate, stearyl benzoate, behenyl isostearate, cetyl myristate, cetyl octanoate, cetyl oleate, cetyl ricinoleate, cetyl stearate, decyl oleate, di-C₁₂-C₁₅alkyl fumerate, dibehenyl fumerate, myristyl lactate, myristyl lignocerate, myristyl myristate, myristyl stearate, lauryl stearate, octyldodecyl stearate, octyldodecyl stearoyl stearate, oleyl arachidate, oleyl stearate, tridecyl behenate, tridecyl stearoyl stearate, pentaerythrityl tetrabehenate, pentaerythritylhydrogenated rosinate, pentaerythrityl distearate, pentaerythrityl tetraabeite, pentaerythrityl tetracocoate, pentaerythrityl tetraperlargonate, pentaerythrityl tetrastearate, ethylene vinyl acetate, polyethylene, hydrogenated cottonseed oil, hydrogenated vegetable oil, hydrogenated squalene, hydrogenated coconut oil, hydrogenated jojoba oil, hydrogenated palm oil, hydrogenated palm kernel oil, hydrogenated olive oil, polyamides, metal stearates and other metal soaps, C₃₀-C₆₀ fatty alcohols, C₂₀+ fatty amides, polypropylene, polystyrene, polybutane, polybutylene terephthalate, polydipentane, polypropylene, zinc stearate, dodecyl laurate, stearyl palmitate, octadecyl hexadecanoate, octadecyl palmitate, stearyl behenate, docosyl octanoate, tetradecyl-octadecanyl behenate, hexadecyl-cosanyl hexacosanate, shellac wax, glycol montanate, fluoranated waxes, C₂₀-C₄₀ alkyl hydroxystearyl stearate and mixtures of such compounds. Examples of suitable branched esters include tetradecyl-octadecanyl behenate and hexadecyl-cosanyl-hexacosanate.

In one embodiment, the present transdermal joint pain therapy composition includes an additional emulsifying agent, such as isopropyl myristate that has the chemical formula of CH₃(CH₂)₁₂COOCCH(CH₃)₂, (CAS # 110-27-0) and isopropyl palmitate that has the chemical formula of CH₃(CH₂)₁₄COOCH(CH₃)₂, (CAS # 142-91-6). In one embodiment, the present transdermal joint pain therapy composition contains isopropyl myristate in an amount preferably between 0% to 20% by weight, based on the total weight of the transdermal joint pain therapy composition. A non-limiting exemplary chemical structure for isopropyl myristate is:

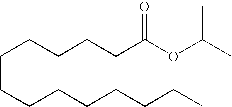

Other exemplary emulsifiers include phosphatidyl choline, phosphatidyl serine, phosphatidyl ethanolamine, phosphatidyl inositol, phosphatidyl glycerol, sphingomyelin, soybean lecithin, corn lecithin, cotton seed oil lecithin, egg yolk lecithin, egg white lecithin, etc.; hydrogenated lecithins; and phospholipid derivatives as formed by introducing polyethylene glycol or aminoglycans into those phospholipids. One or more of those phospholipids may be in the composition. Of those phospholipids, preferred are soybean lecithin, egg yolk lecithin, hydrogenated soybean lecithin, and hydrogenated egg yolk lecithin.

The present transdermal joint pain therapy composition may also include additional emollients and emulsifiers, such as long-chain saturated fatty alcohols, such as behenyl alcohol. Other additional exemplary emollients and emulsifiers include of C₁₄-C₂₂ fatty alcohols, C₁₂-C₂₂ fatty acids, and C₁₂-C₂₂ fatty alcohol ethoxylates having an average degree of ethoxylation ranging from 2 to about 30, and mixtures thereof. Preferred immobilizing agents include C₁₆-C₁₈ fatty alcohols, most preferably crystalline high melting materials selected from the group consisting of cetyl alcohol, stearyl alcohol, and mixtures thereof.

In one embodiment, the present transdermal joint pain therapy composition includes an anti-oxidant agent, such as butylated hydroxytoluene (BHT) that has the chemical formula of 2,6-Bis(1,1-dimethylethyl)-4-methylphenol, (CAS # 128-37-0). BHT is a fat-soluble compound that is a crystalline phenolic antioxidant preservative. Butylated hydroxytoluene is readily absorbed from the gastrointestinal tract; it is excreted in the uring mainly as glucuronide conjugates of oxidation products. In one embodiment, the present transdermal joint pain therapy composition contains BHT in an amount preferably between 0% to 2% by weight, based on the total weight of the transdermal joint pain therapy composition.

A non-limiting exemplary chemical structure for urea is:

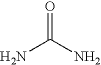

In one embodiment, the present transdermal joint pain therapy composition includes a mold or yeast growth inhibitor, such as potassium sorbate that has the chemical formula C₆H₇O₂K, (CAS # 590-00-1). Potassium sorbate, the organic salt of sorbic acid, contains not less than 98 percent and not more than the equivalent of 102 percent of C₆H₇O₂K. Generally, potassium sorbate is used as an antimicrobial or fungistatic agent. In one embodiment, the present transdermal joint pain therapy composition contains potassium sorbate in an amount preferably between 0% to 0.5% by weight, based on the total weight of the transdermal joint pain therapy composition. A non-limiting exemplary chemical structure for potassium sorbate is:

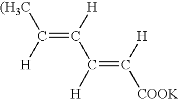

In one embodiment, the present transdermal joint pain therapy composition includes alkalizing agents for adjusting the pH of the transdermal joint pain therapy composition, such as sodium hydroxide that has the chemical formula NaOH, (CAS # 1310-73-2). Sodium hydroxide is a strong base that is highly soluble in water. In one embodiment, the present transdermal joint pain therapy composition contains sodium hydroxide in an amount preferably between 0% to 0.5% by weight, based on the total weight of the transdermal joint pain therapy composition.

The present transdermal joint pain therapy composition may also include additional alkalizing agents. Nonlimiting examples of such alkalizing agents include potassium hydroxide, ammonium hydroxide, monethanolamine, diethanolamine, triethanolamine, diisopropanolamine, aminomethylpropanol, tromethamine, tetrahydroxypropyl ethylenediamine, and mixtures thereof.

In one embodiment, the present transdermal joint pain therapy composition includes chelating agents for chelating oxidizing agents of the transdermal joint pain therapy composition, such as ethylenediaminitetraacetic acid (EDTA) disodium (CAS # 139-33-3). In one embodiment, the present transdermal joint pain therapy composition contains EDTA disodium in an amount preferably between 0% to 5% by weight, based on the total weight of the transdermal joint pain therapy composition. A non-limiting exemplary chemical structure for EDTA disodium is:

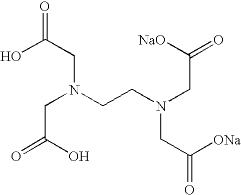

In one embodiment, the present transdermal joint pain therapy composition includes an anti-oxidizing agent of the transdermal joint pain therapy composition, such as lecithin granules, (CAS # 8002-43-5). In one embodiment, the present transdermal joint pain therapy composition contains lecithin granules in an amount preferably between 0% to 10% by weight, based on the total weight of the transdermal joint pain therapy composition.

Two non-limiting exemplary chemical structures for lecithin is:

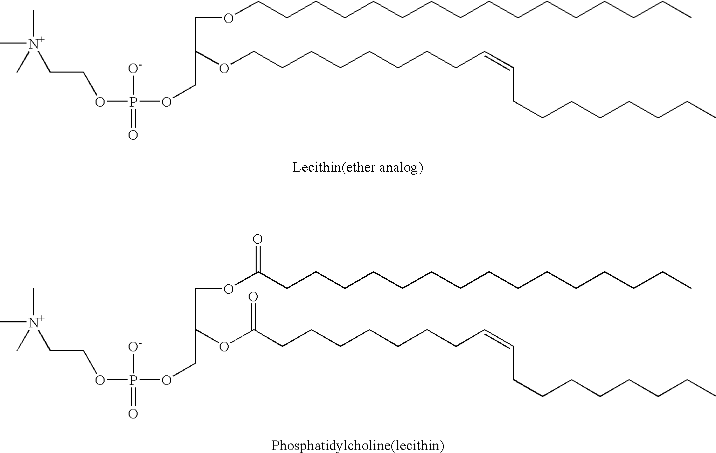

In one embodiment, the present transdermal joint pain therapy composition includes water. Preferably, the water includes a water-soluble preservative, such a paraben. Preferably, exemplary parabens are selected among methylparabens, ethylparabens, propylparabens, and butylparabens. Parabens are also known by other names, such as esters of p-hydroxybenzoic acid. One or more of these parabens may be selected and combined for use in the present transdermal joint pain therapy composition. Another exemplary preservative of the present transdermal joint pain therapy composition is Phenonip™, which is a tradename for a mixture of paraben compounds made by Clariant and it contains the following components: phenoxyethanol (CAS # 122-99-6), ethylparaben (CAS # 99-76-3), butylparaben (CAS # 94-26-8), ethylparaben (CAS # 120-47-8), and propylparaben (CAS # 94-13-3). In addition to being a preservative, these preservatives further provide microbial contamination of the present transdermal joint pain therapy composition and are effective against Gram-positive and Gram-negative bacteria, yeasts, and molds. In one embodiment, the present transdermal joint pain therapy composition contains water in an amount preferably between 10% to 95% by weight, based on the total weight of the transdermal joint pain therapy composition. Also, the present transdermal joint pain therapy composition contains a preservative in an amount preferably between 0.1% to 2% by weight, based on the total weight of the transdermal joint pain therapy composition. Additionally, these amounts of water and preservatives can be increased or decreased as desired.

In one embodiment, the transdermal base according to the present transdermal joint pain therapy composition includes propylene glycol, cetyl alcohol, stearyl alcohol, stearic acid, glyceryl monostearate, isopropyl myristate, lecithin granules, isopropyl palmitate, butylated hydroxytoluene, simethicone, urea, potassium sorbate, sodium hydroxide, polyoxy 40 stearate, EDTA disodium, and water. In another embodiment, the transdermal base according to the present transdermal joint pain therapy composition includes propylene glycol, cetyl alcohol, stearic acid, glyceryl monostearate, isopropyl myristate, lecithin granules, isopropyl palmitate, butylated hydroxytoluene, simethicone, urea, potassium sorbate, sodium hydroxide, polyoxy 40 stearate, EDTA disodium, and water.

### Joint Pain Therapy Component

In one embodiment, the present transdermal joint pain therapy composition includes glutamine, glucosamine, hyaluronic acid, and dimethyl sulfone. In another embodiment, the present transdermal joint pain therapy composition includes glutamine, glucosamine, hyaluronic acid, glucosamine HCl, and dimethyl sulfone.

In one embodiment, the present transdermal joint pain therapy composition includes glutamine that has the chemical formula NH₂CO(CH₂)₂CHNH₃CO₂, (CAS # 6899-04-3). Glutamine is the amide of glutamic acid, and is uncharged under all biological conditions. It is an amino acid that is known for its properties as an amine donor to other active biological compounds. It is known that glutamine contributes amine to glucose for the formation of glucosamine, which is a substance found in the synovial fluid that draws water to itself, creating a nourishing and pressurized capsular joint. Glutamine is also known as aminoglutaramic acid, oxopentanoic, flutamic acid amide, Cebrogen, Glumin, and Levoglutaminia.

In this embodiment, the present transdermal joint pain therapy composition contains glutamine in an amount preferably between 2.5% to 15.0% by weight, based on the total weight of the transdermal joint pain therapy composition. A non-limiting exemplary chemical structure for glutamine is:

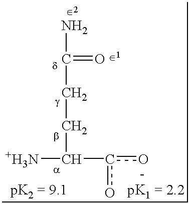

In one embodiment, the present transdermal joint pain therapy composition includes glucosamine HCl (CAS # 3416-24-8). Glucosamine is also known as Dona, Chitosamine, and amino deoxy glucose. In this embodiment, the present transdermal joint pain therapy composition contains glucosamine HCl in an amount preferably between 0% to 15.0% by weight, based on the total weight of the transdermal joint pain therapy composition.

Glucosamine HCl is one of the building blocks of several glycosaminoglycans including keratin sulfates I and II, hyaluronic acid, heparin, and heparin sulfate. The latter two glycosaminoglycans utilize glucosamine in its non-acetylated state, whereas the former three utilize glucosamine in it acetylated state, N-acetylglucosamine. Glycoaminoglycans are the major components of mucous, the bodies ground substance, and with much importance to synovial fluid. The structure of glycoaminoglycans is a long, unbranched, heteropolysaccharide composed of repeating disaccharide units. These disaccharides are composed of one acidic sugar (either D-glucosamine or D-galactosamine). These negative charges, as well as the sulfate groups found abundantly in glycosaminoglycans, give these molecules their strong negative charge. It is this negative charge that imparts glycosaminoglycans with their functional properties. These aforementioned negative charges, and therefore slide past one another much like magnets of similar polarity slide past one another. When compressed the glycosaminoglycans give up water molecules. This property allows for the resilience seen in synovial fluid when pressure is placed on the joint. A non-limiting exemplary chemical structure for glucosamine HCl is:

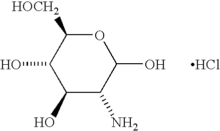

In one embodiment, the present transdermal joint pain therapy composition includes hyaluronic acid (CAS # 9004-61-9). Hyaluronic acid is a vital component of the extracellular fluid matrix. Retention of water is one of the most important biological functions of hyaluronic acid, second only to providing nutrients and removing waste from cells that do not have a direct blood supply, such as cartilage cells. Hyaluronic acid forms the backbone of the essential molecules of the joint matrix, proteoglycan aggregates. These molecules allow for the lubrication and tensile strength necessary for proper joint function. With an insufficient amount of hyaluronic acid, nutrients cannot be moved into these cells and waste cannot be eliminated. Hyaluronic acid is also known as Hyacid, ARTZ, Connettiva, Equron, Healon, Healonid, Hyalgan, Hyalovet, Ial, Opegan, Proyisc, and Synacid. In this embodiment, the present transdermal joint pain therapy composition contains hyaluronic acid in an amount preferably between 0.04% to 5% by weight, based on the total weight of the transdermal joint pain therapy composition.

A non-limiting exemplary chemical structure for hyaluronic acid is:

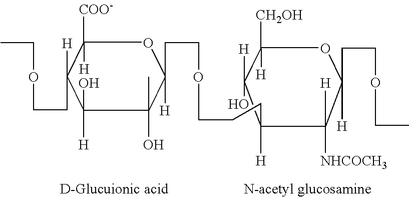

In one embodiment, the present transdermal joint pain therapy composition includes methylsulfonylmethane (MSM) that has a chemical formula of CH₃SO₂CH₃, (CAS #67-71-0). MSM is a naturally occurring, odorless breakdown product of dimethyl sulfoxide (DMSO). MSM is purported to have anti-inflammatory and anti-cancer properties and is used to inhibit prostacyclin (PG12) synthesis in cultured cells of the endothelium, an action that is believed to combat atherosclerosis. Another use of MSM is to combat osteoarthritis of the knee. MSM is also known as dimethyl sulfate and dimethylsulfone (DMSO₂), which reflects its close metabolic relationship to DMSO. In this embodiment, the present transdermal joint pain therapy composition contains MSM in an amount preferably between 2.5% to 10.0% by weight, based on the total weight of the transdermal joint pain therapy composition. A non-limiting exemplary chemical structure for MSM is:

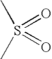

The present transdermal joint pain therapy composition may also include additional water-soluble components, such as alcohols; humectants, including polyhydric alcohols (e.g. glycerine and propylene glycol); active agents such as d-panthenol, vitamin B₃ and its derivatives (such as niacinamide) and botanical extracts; thickeners and preservatives.

The present transdermal joint pain therapy composition may also include gelling agents, such as carboxyvinyl polymers, acrylic copolymers such as acrylate/alkylacrylate copolymers, polyacrylamides, polysaccharides, such as hydroxypropylcellulose, natural gums and clays, and, as lipophilic gelling agents, representative are the modified clays such as bentones, fatty acid metal salts such as aluminum stearates and hydrophobic silica, or ethylcellulose and polyethylene.

The present transdermal joint pain therapy composition may also include at least one oil, such as octyldecyl myristate. Other exemplary oils include hydrocarbon-based oils such as liquid paraffin or liquid petroleum jelly, mink oil, turtle oil, soybean oil, perhydrosqualene, sweet almond oil, beauty-leaf oil, palm oil, grapeseed oil, sesame seed oil, corn oil, parleam oil, arara oil, rapeseed oil, sunflower oil, cottonseed oil, apricot oil, castor oil, avocado oil, jojoba oil, olive oil or cereal germ oil; esters of lanolic acid, of oleic acid, of lauric acid or of stearic acid; fatty esters, such as isopropyl myristate, isopropyl palmitate, butyl stearate, hexyl laurate, diisopropyl adipate, isononyl isononate, 2-ethylhexyl palmitate, 2-hexyldecyl laurate, 2-octyldecyl palmitate, 2-octyldodecyl myristate or lactate, 2-diethylhexyl succinate, diisostearyl malate, glyceryl triisostearate or diglyceryl triisostearate; higher fatty acids such as myristic acid, palmitic acid, stearic acid, behenic acid, oleic acid, linoleic acid, linolenic acid or isostearic acid; higher fatty alcohols such as cetanol, stearyl alcohol or oleyl alcohol, linoleyl alcohol or linolenyl alcohol, isostearyl alcohol or octyidodecanol; silicone oils such as polydimethylsiloxanes (PDMS), which are optionally phenylated such as phenyltrimethicones, or optionally substituted with aliphatic and/or aromatic groups that are optionally fluorinated, or with functional groups such as hydroxyl, thiol and/or amine groups; polysiloxanes modified with fatty acids, with fatty alcohols or with polyoxyalkylenes, fluorosilicones and perfluoro oils.

A non-limiting exemplary chemical structure for BHT is:

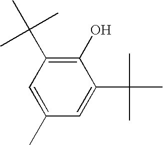

### 2,6-Bis(1,1-dimethylethyl)-4-methylphenol

In one embodiment, the present transdermal joint pain therapy composition includes an anti-flatulence agent, such as simethicone, (CAS # 8050-81-5). In one embodiment, the present transdermal joint pain therapy composition contains simethicone in an amount preferably between 0% to 3% by weight, based on the total weight of the transdermal joint pain therapy composition. A non-limiting exemplary chemical structure for simethicone is:

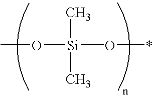

In one embodiment, the present transdermal joint pain therapy composition includes an emolient, such as urea that has the chemical formula CO(NH₂)₂ (CAS # 57-13-6). Urea is a water-soluble compound that is the major nitrogenous end product of protein metabolism. Urea is normally cleared from the blood by the kidney into the urine. Urea is used in topical dermatological products to promote rehydration of the skin. In one embodiment, the present transdermal joint pain therapy composition contains urea in an amount preferably between 0% to 15% by weight, based on the total weight of the transdermal joint pain therapy composition.

The transdermal joint pain therapy composition preferably may also include additional emollients, such as squalane. Other exemplary emollients include castor oil, polybutene, sweet almond oil, avocado oil, calophyllum oil, ricin oil, vitamin E acetate, olive oil, silicone oils such as dimethylopolysiloxane and cyclomethicone, linolenic alcohol, oleyl alcohol, the oil of cereal germs such as the oil of wheat germ, isopropyl palmitate, octyl palmitate, isopropyl myristate, hexadecyl stearate, butyl stearate, decyl oleate, acetyl glycerides, the octanoates and benzoates of (C₁₂-C₁₅) alcohols, the octanoates and decanoates of alcohols and polyalcohols such as those of glycol and glyceryl, ricinoleates esters such as isopropyl adipate, hexyl laurate and octyl dodecanoate, dicaprylyl maleate, hydrogenated vegetable oil, phenyltrimethicone, jojoba oil and aloe vera extract.

The transdermal joint pain therapy composition preferably may also include viscosity builders, such as cetearyl alcohol and polysorbate 60. Other exemplary viscosity builders include ceteareth-25 and ceteareth-6, i.e., polyethylene glycol ethers of cetearyl alcohol with 25 and 6 ethylene glycol units respectively.

The present transdermal joint pain therapy composition may also include skin penetrating agents such as tribehenin. Other exemplary penetrating agents include waxes which are solid or semi-solid at room temperature, such as animal waxes, plant waxes, mineral waxes, silicone waxes, synthetic waxes, and petroleum waxes. More specifically, these waxes include bayberry, beeswax, candelilla, carnauba, ceresin, cetyl esters, hydrogenated jojoba oil, hydrogenated jojoba wax, hydrogenated microcrystalline wax, hydrogenated rice bran wax, japan wax, jojoba butter, jojoba esters, jojoba wax, lanolin wax, microcrystalline wax, mink wax, montan acid wax, montan wax, ouricury wax, ozokerite, paraffin, cetyl alcohol, beeswax, PEG-20 sorbitan beeswax, PEG-8 beeswax, rice bran wax, shellac wax, spent grain wax, sulfurized jojoba oil, synthetic beeswax, synthetic candelilla wax, synthetic carnauba wax, synthetic japan wax, synthetic jojoba oil, synthetic wax, polyethylene, stearoxy dimethicone, dimethicone behenate, stearyl dimethicone, and the like, as well synthetic homo- and copolymer waxes such as PVP/eicosene copolymer, PVP/hexadecene copolymer, and the like.

In one embodiment, the transdermal joint pain includes glutamine, glucosamine, hyaluronic acid, dimethyl sulfone, propylene glycol, cetyl alcohol, stearyl alcohol, stearic acid (triple pressed), glyceryl monostearate (pure), isopropyl myristate, lecithin granules, isopropyl palmitate, butylated hydroxytoluene, simethicone, urea, potassium sorbate, sodium hydroxide (30% solution), polyoxy 40 stearate, EDTA disodium, and water (USP, preserved with parabens). It is applied in a cream or gel form to the skin area around a joint of a subject.

In addition to the aforementioned aspects and embodiments of the present invention, the present invention further includes methods for treating joint pain. The present method for treating joint pain includes applying the transdermal joint pain composition to the skin approximately adjacent to an affected joint of a subject. The application amount can be any volume desired, and in one embodiment it is 2 mls. In this embodiment, the 2 mls of transdermal joint pain composition is applied twice daily to the skin of the affected joint. Preferably, the transdermal joint pain composition is massaged into the skin for approximately 3 minutes, although this duration can be extended or shortened as desired. In addition, heat or ultrasound may be applied post-massage to the skin area by a health care professional. An exemplary ultrasound method includes using an ultrasound machine on the affected joint for a duration of 5 minutes. Depending on the treatment, preferably the ultrasound applications may be daily or several times per week. An exemplary ultrasound machine is a Dynatron 950 Plus that is operated with a non-pulsating, continuous setting at a range of 2-3 Mhz with the intensity set at 1.5 W/cm².

The following tables and examples further describe and demonstrate embodiments within the scope of the present invention. The examples are given solely for the purpose of illustration and are not to be construed as limitations of the present invention, as many variations thereof are possible without departing from the spirit and scope of the invention. Ingredients are identified by chemical or CTFA name, or otherwise defined below. Unless otherwise noted, all amounts are percentages by weight based on the total composition weight.

TABLE 1
Transdermal Formulation
Exemplary Chemical Formulation

| Ingredient | Quantity | |
|---|---|---|
| Cetyl Alcohol | 10.0 | gms |
| Stearyl Alcohol | 5.0 | gms |
| Stearic Acid Triple Pressed | 50.0 | gms |
| Glyceryl Monostearate Pure | 50.0 | gms |
| Isopropyl Myristate Cosmetic | 125.0 | gms |
| Lecithin/Isopropyl Palm. Soln. | 66.0 | mls |
| Butylated HydroxylToluene NF | 10 | mls |
| Simethicone | 20.0 | gms |
| Urea | 100.0 | gms |
| Potassium Sorbate | 2.0 | gms |
| Sodium Hydroxide 30% Solution | 2.5 | mls |
| Polyoxyl 40 Stearate Polyox | 50.0 | gms |
| EDTA Disodium | 20.0 | mls |
| Water | qs 1,000.0 | gms |

Another embodiment of the composition of the transdermal formulation is shown in Table 2.

TABLE 2
Transdermal Formulation
Exemplary Chemical Formulation

| Ingredient | Quantity | |
|---|---|---|
| Cetyl Alcohol | 15.0 | gms |
| Stearic Acid Triple Pressed | 50.0 | gms |
| Glyceryl Monostearate Pure | 50.0 | gms |
| Isopropyl Myristate Cosmetic | 125.0 | gms |
| Lecithin/Isopropyl Palm. Soln. | 66.0 | mls |
| Butylated HydroxylToluene NF | 10 | mls |
| Simethicone | 20.0 | gms |
| Urea | 100.0 | gms |
| Potassium Sorbate | 2.0 | gms |
| Sodium Hydroxide 30% Solution | 2.5 | mls |
| Polyoxyl 40 Stearate Polyox | 50.0 | gms |
| EDTA Disodium | 20.0 | mls |
| Water | qs 1,000.0 | gms |

### EXAMPLE 1

### Preparation of Transdermal Formulation

The preparation of 1,000 gms of Transdermal Formulation of Tables 1 and 2 are as follows. Cetyl Alcohol (if used), Stearyl Alcohol, Stearic Acid, and Glyceryl Monostearate are placed into a first vessel and are heated to approximately 60° C.-70° C. Then isopropyl myristate, lecithin/isopropyl palmitate, BHT 10%/alcohol solution, and simethicone are added to the first vessel with stirring. In a second vessel, dissolve potassium sorbate, urea, and poloxyl 40 stearate in water, preserved that is approximately 30% of the final weight. Add heated sodium hydroxide 30% and EDTA 5% to the second vessel with constant mixing. Add contents of the second vessel to the first vessel with constant stirring. Heat water to approximately 60° C.-70° C.; then add this water to the first vessel until 1000 gms of total mixture is produced. Adjust pH between 6.5 and 6.8 with sodium hydroxide solution. Cease heating the first vessel, but continue mixing until gel thickens and is uniform.

TABLE 3
Transdermal Joint Pain Composition
Exemplary Chemical Formulation

| Ingredient | Quantity | |
|---|---|---|
| Glutamine | 100.0 | gms |
| Glucosamine HCl | 100.0 | gms |
| Hyaluronic Acid Sodium Salt | 2.5 | gms |
| Dimethyl Sulfone | 87.5 | gms |
| Lecithin Organogel | 166.7 | mls |
| Propylene Glycol | 166.7 | mls |
| Example1 Transdermal Formulation | 1,000 | mls |

Another embodiment of the composition of the transermal join pain formulation is shown in Table 4.

TABLE 4
Transdermal Joint Pain Composition
Exemplary Chemical Formulation

| Ingredient | Quantity | |
|---|---|---|
| Glutamine | 100.0 | gms |
| Glucosamine HCl | 100.0 | gms |
| Hyaluronic Acid Sodium Salt | 4.2 | gms |
| Dimethyl Sulfone | 87.5 | gms |
| Lecithin Organogel | 166.7 | mls |
| Propylene Glycol | 166.7 | mls |
| Example 1 Transdermal Formulation | 1,000 | mls |

### EXAMPLE 2

### Preparation of Transdermal Joint Pain Formulation

The preparation of 1,000 mls of the Transdermal Joint Pain Formulation as described in Tables 3 and 4 are as follows. Dissolve the hyaluronic acid in an adequate amount of preserved water. An approximate amount of water is 20 mls per 1 gm of hyaluronic acid. In addition, the water is heated for improved dissolution. Weigh and mix the glutamine, glucosamine HCl and dimethyl sulfone and then place one half of the mixture into a first reaction vessel and add the propylene glycol. Place the other half of the mixture into a second reaction vessel and add the Lecithin Organel. While mixing, add hyaluronic acid solution to the first reaction vessel containing the propylene glycol mixture. While mixing, add the Lecithin Organel mixture to the first reaction vessel. Add the transdermal formulation from Example 1 to bring total volume to 1,000 mls. Place total mixture into an ointment mill. The milling serves to smooth the texture of the cream. Preferably, the total volume of 1,000 mls is used to make smaller-volumed individual treatment doses, such as 2 mls.

### Clinical Trial

A prospective, controlled, randomized, double-blind clinical trial was conducted using the transdermal joint pain therapy composition of Table 3. The trial includes two groups of subjects, Group II subjects received the transdermal joint pain composition of Example 2 and Group I subjects received a placebo. All subjects were recruited under the discretion of a principal investigator with the following inclusion criteria: subject symptoms (daily pain, pain restricts work, recreation and/or ADL, stiffness of the knee, and instability of the knee); cognitive function sufficient to understand protocol and to complete subject diary or other analysis tools employed; must read, write, and understand English language; ASA risk 1 or 2; provided written informed consent. Exclusion criteria consisted of: neuropathic joint; age and functional demands/activity level; knee sepsis including previous osteomyelitis; remote source of ongoing sepsis; severe vascular disease; comorbid conditions preventing full functional activity or which requires continuous use of pain medication; known history of allergy, sensitivity or any other form of reaction to the ingredients; suspected inability to comply with study procedures, including language difficulties or medical history and/or concomitant disease, as judged by the investigator; neurological and/or vascular condition which may affect the outcome of the procedure; receiving regular treatment with analgesics, sedatives, or any other medication with central nervous system effects; women who are pregnant or are ot practicing medically acceptable contraception; and participation in other clinical studies during this trial or in the 14 days prior to the admission to this study.

The primary outcome measure for this trial was a Subject Pain Diary and knee range of motion. The subjects recorded in the diary their subjective evaluations of pain, comfort, ability to sleep, activity level, and quality of life. Knee range of motion was measured prior to treatment and at two weeks of treatment.

The treatment included topical application twice daily of the transdermal joint pain therapy composition to the skin area adjacent to the affected knee of the subjects. 24 subjects were enlisted and 2 withdrew from the study. The median age of the subjects was 46.5 and the mean age of the subjects was 41.5. The Group I subjects were administered a placebo twice daily, while the Group II subjects were administered the present transdermal joint pain therapy. Group I consisted of 10 subjects (4 male and 6 female) that had a mean age of 42.5 and range of motion of +6. Table 5 summarizes the data from the clinical trial

TABLE 5
Clinical Trial

| | | | | ROM | ROM | |
|---|---|---|---|---|---|---|
| Group | Knee | Age | Gender | Pre-Trial | Post-Trial | Scale |
| I | R | 56 | F | 0-100 | N/A | N/A |
| I | R | 14 | M | 0-120 | 0-135 | Slight worsening |
| I | R | 27 | F | 0-130 | 0-140 | Marked improvement |
| I | R | 49 | F | 0-90 | 0-100 | Same |
| I | L | 50 | M | 0-130 | 0-120 | Slight improvement |
| I | L | 51 | F | 0-100 | 0-120 | Same |
| I | R | 56 | F | 0-90 | 0-120 | Improvement |
| I | L | 46 | F | 0-110 | 0-120 | Same |
| I | L | 31 | M | 0-120 | 0-125 | Slight improvement |
| I | R | 52 | M | 0-130 | 0-120 | Same |
| I | R | 49 | F | 0-120 | 0-110 | No improvement |
| II | R | 50 | F | 0-120 | 0-120 | Same |
| II | L | 44 | M | 0-120 | 0-130 | Slight improvement |
| II | R | 23 | M | 0-120 | 0-120 | Same |
| II | R | 18 | M | 0-120 | 0-130 | Same |
| II | R | 17 | M | 0-130 | 0-140 | Improvement |
| II | R | 47 | M | 0-120 | 0-140 | Improvement |
| II | L | 27 | M | 0-100 | 0-120 | Improvement |
| II | R | 63 | F | 0-120 | 0-120 | Slight improvement |
| II | R | 55 | M | 0-120 | 0-120 | Same |
| II | L | 36 | M | 0-120 | 0-130 | Improvement |
| II | R | 58 | M | 0-100 | 0-120 | Same or worse/high activity level |
| II | L | 51 | M | 0-90 | 0-110 | Slight improvement |

Table 6 summarizes the results from Table 5 showing a significant improvement of Group II compared to Group I.

TABLE 6
Clinical Trial

| Pain Scale | Group I | Group II |
|---|---|---|
| Slight improvement | 20% | 25% |
| Improvement | 10% | 33.33% |
| Marked improvement | 10% | 0% |
| Same | 50% | 33.33% |
| Slightly worse | 10% | 8.33% |
| Worse | 0% | 0% |
| Marked worsening | 0% | 0% |
| Reported some level of improvement | 40% | 58.33% |
| Reported staying the same | 50% | 33.33% |
| Reported some level of worsening | 10% | 8.33% |

Although there has been described what is at present considered to be the preferred embodiments of the composition and methods for transdermal joint pain therapy, it will be understood that the present transdermal joint pain therapy can be embodied in other specific forms without departing from the spirit or essential characteristics thereof. For example, additional transdermal delivery agents, other than those described herein, could be used without departing from the spirit or essential characteristics of the present composition and methods for transdermal joint pain therapy. The present embodiments are, therefore, to be considered in all aspects as illustrative and not restrictive. The scope of the invention is indicated by the appended claims rather than the foregoing description.

What is claimed:

1. A transdermal joint pain therapy composition comprising:
(a) from about 2.5% to about 15%, based on the total weight of said transdermal joint pain therapy composition, of glutamine;
(b) from about 0.04% to about 0.5%, based on the total weight of said transdermal joint pain therapy composition, of hyaluronic acid;
(c) from about 2.5% to about 10.0%, based on the total weight of said transdermal joint pain therapy composition, of methylsulfonylmethane; and
(d) from about 70% to about 95%, based on the total weight said transdermal joint pain therapy composition, of a transdermal delivery agent.

2. The transdermal joint pain therapy composition of claim 1, wherein said transdermal delivery agent comprises:
at least one or more compounds selected from the group consisting of cetyl alcohol, stearyl alcohol, stearic acid, glyceryl monostearate, isopropyl myristate, lecithin, butylated hydroxyl toluene, simethicone, urea, potassium sorbate, sodium hydroxide, polyoxyl 40 stearate, EDTA disodium, and water.

3. The transdermal joint pain therapy composition of claim 1, further comprising:
from about 0.1% to about 15.0%, based on the total weight of the composition, of glucosamine HCl.

4. The transdermal joint pain therapy composition of claim 1, further comprising:
from about 5.0% to about 20.0%, based on the total weight of the composition, of lecithin.

5. The transdermal joint pain therapy composition of claim 1, further comprising:
from about 5.0% to about 20.0%, based on the total weight of the composition, of propylene glycol.

6. The transdermal joint pain therapy composition of claim 1, wherein said transdermal delivery agent further comprises:
a paraben based preservative.

7. The transdermal joint pain therapy composition of claim 6, wherein said paraben-based preservative comprises:
from about 0.6% to about 0.9%, based on the total weight of the transdermal joint pain therapy composition, of a mixture selected from the group consisting of phenoxyethanol, methylparaben, propylparaben, butylparaben, and isobutylparaben.

8. The transdermal joint pain therapy composition of claim 1, wherein said transdermal delivery agent comprises a mixture selected from the group consisting of a gel, a cream, and an ointment.

9. A transdermal joint pain therapy composition comprising:
(a) from about 2.5% to about 15%, based on the total weight of said transdermal joint pain therapy composition, of glutamine;
(b) from about 0.04% to about 0.5%, based on the total weight of said transdermal joint pain therapy composition, of hyaluronic acid;
(c) from about 2.5% to about 10.0%, based on the total weight of said transdermal joint pain therapy composition, of methylsulfonylmethane;
(d) from about 0.1% to about 15.0%, based on the total weight of the composition, of glucosamine HCl;
(e) from about 5.0% to about 20.0%, based on the total weight of the composition, of lecithin;
(f) from about 5.0% to about 20.0%, based on the total weight of the composition, of propylene glycol; and
(g) from about 70% to about 95%, based on the total weight said transdermal joint pain therapy composition, of at least one or more compounds selected from the group consisting of cetyl alcohol, stearyl alcohol, stearic acid, glyceryl monostearate, isopropyl myristate, lecithin, butylated hydroxyl toluene, simethicone, urea, potassium sorbate, sodium hydroxide, polyoxyl 40 stearate, EDTA disodium, and water.

10. The transdermal joint pain therapy composition of claim 9, wherein said transdermal joint pain therapy composition comprises a mixture selected from the group consisting of a gel, a cream, and an ointment.

11. A method for treating pain of an affected joint of a subject comprising: providing a transdermal joint pain therapy composition comprising:
(a) from about 2.5% to about 15%, based on the total weight of said transdermal joint pain therapy composition, of glutamine;
(b) from about 0.04% to about 0.5%, based on the total weight of said transdermal joint pain therapy composition, of hyaluronic acid;
(c) from about 2.5% to about 10.0%, based on the total weight of said transdermal joint pain therapy composition, of methylsulfonylmethane;
(d) from about 70% to about 95%, based on the total weight said transdermal joint pain therapy composition, of a transdermal delivery agent;
applying said transdermal joint pain therapy composition to said skin area adjacent to said affected joint; and
massaging said transdermal joint pain therapy composition into said skin area.

12. The method for treating pain of an affected joint of a subject of claim 11 wherein said applying said transdermal joint pain therapy composition to said skin area includes applying twice per day said transdermal joint pain therapy composition to said skin area.

13. The method for treating pain of an affected joint of a subject of claim 11, further comprising:
applying heat to said skin after said massaging said transdermal joint pain therapy composition into said skin area.

14. The method for treating pain of an affected joint of a subject of claim 11, further comprising:
applying ultrasound to said skin after said massaging said transdermal joint pain therapy composition into said skin area.

15. The method for treating pain of an affected joint of a subject of claim 11 wherein said applying said transdermal joint pain therapy composition to said skin area adjacent to said affected joint is done for a duration of 1 to 10 minutes.

16. The method for treating pain of an affected joint of a subject of claim 11 wherein said applying said transdermal joint pain therapy composition to said skin area adjacent to said affected joint is done for a duration of 3 minutes.

17. A method of preparation of a transdermal joint pain therapy composition comprising:
(a) preparing a first mixture comprising:
(a1) heating cetyl alcohol, stearyl alcohol, stearic acid, and glyceryl monostearate in a first vessel at a temperature of approximately 60° C.-70° C.;
(a2) mixing isopropyl myristate, lecithin/isopropyl palmitate, BHT 10%/alcohol solution, and simethicone into said first vessel;
(b) preparing a second mixture comprising:
(b1) dissolving potassium sorbate, urea, and poloxyl 40 stearate in water;
(c) adding said heated sodium hydroxide and EDTA to said second vessel with constant mixing;
(d) adding the contents of said second vessel to said first vessel constant stirring;
(e) heating said water to approximately 60° C.-70° C. and adding said heated water to said first vessel; and
(f) adjusting the pH between 6.5 and 6.8 of said first vessel with sodium hydroxide solution.

18. The method of preparation of a transdermal joint pain therapy composition of claim 17 further comprising:
enclosing a portion of said transdermal joint pain therapy composition into individual treatment containers.

19. The method of preparation of a transdermal joint pain therapy composition of claim 18 wherein said individual treatment containers have a volume of 2 mls.
